(19) United States
(12) Patent Application Publication
(10) Pub. No.: US 2025/0387569 A1
(43) Pub. Date: Dec. 25, 2025

(54) Magnetic Force-Driven Injection Apparatus

(71) Applicant: ZHEJIANG SUMMED MEDTECH CO., LTD., Huzhou, Zhejiang (CN)

(72) Inventor: Jian Sen WENG, Taoyuan (TW)

(21) Appl. No.: 18/878,857

(22) PCT Filed: Sep. 8, 2022
(86) PCT No.: PCT/CN2022/117695

§ 371 Date: Dec. 24, 2024

(30) Foreign Application Priority Data
Jul. 4, 2022 (CN) 202210780407.5

(51) Int. Cl. A61M 5/20; H02K 11/33; H02K 33/18

(52) U.S. Cl. CPC A61M 5/20; H02K 11/33; H02K 33/18; A61M 2205/02; A61M 2205/8287

(57) ABSTRACT

The present invention discloses a magnetic force-driven apparatus designed to perform injection operations by being installed with a vial. The magnetic force-driven injection apparatus includes a sleeve, a power supply unit, a first actuator, a second actuator, and a magnetic force generating unit. The magnetic force-generating unit is selectively coupled with either the first actuator or the second actuator. When the magnetic force-generating unit generates a magnetic force, it generates either a magnetic attraction or a magnetic repulsion between the first actuator and the second actuator, so the second actuator is driven either toward the vial closely to activate the injection or farther away from the vial after the injection.

### CROSS-REFERENCE TO RELATED APPLICATIONS

This application claims the benefit of PCT/CN2022/117695, filed Sep. 8, 2022, which is incorporated in its entirety by reference herein.

### FIELD OF THE INVENTION

The present invention relates to the technical field of an injection apparatus, particularly relates to a magnetically driven and reusable magnetic force-driven injection apparatus.

### BACKGROUND OF THE INVENTION

When the injection apparatus of the prior art performs the medicine injection procedure, the user needs to continuously apply external force to the push rod to drive the medicine into the human body. With the advancement of medical technology, automatic injection apparatus with auxiliary driving force has been used widely. As long as the user operates the automatic injection apparatus with one hand, the push rod can be driven to move to complete the medicine injection, and the push rod can even be returned to the position before injection to reuse the automatic injection apparatus, whereby the convenience and flexibility of injection can be improved.

Most current automatic injection apparatus use springs or motors as the sources of driving force to move the push rod. However, when the driving force of an automatic injection apparatus is provided by springs, the structural configuration and engagement design of multiple components within the automatic injection apparatus must work together to complete the fixing and retraction of the push rod after its movement. The structural assembly is often overly complex. Furthermore, when the driving force of an automatic injection apparatus is provided by motors, a screw mechanism is required to drive the movement of the push rod. On one hand, it requires an additional power supply for the motor's operation. On the other hand, the setting of the motor makes it difficult to reduce the size and weight of the automatic injection apparatus, leading to increased manufacturing costs.

Therefore, simplifying the structure of the assembled parts and more effectively driving the push rod to move back and forth to facilities the user's operation is indeed an important issue.

### SUMMARY OF THE INVENTION

The present invention is designed to address the above technical problems, with the purpose of providing a magnetic force-driven injection apparatus. This apparatus utilizes changes in magnetic force as the driving force to perform injection operations on or away from the medicine, simplifying the structural design and achieving reusability.

The magnetic force-driven injection apparatus of the present includes a sleeve, a power supply unit, a first actuator, a second actuator, and a magnetic force-generating unit. The sleeve includes a channel. The power supply unit coupled with the sleeve. The first actuator is disposed in the channel. The second actuator is movably disposed in the channel, and the second actuator is capable of moving along an axis corresponds to the first actuator. The magnetic force-generating unit is electrically connected to the power supply unit, and the magnetic force-generating unit is selectively coupled with the first actuator or the second actuator. The power supply unit providing the unit supply current to the magnetic force-generating unit, so a magnetic attraction or a magnetic repulsion is created between the first actuator and the second actuator, so the second actuator is driven either to the vial closely to activate the injection or farther away from the vial after the injection.

In one preferred embodiment, the magnetic force-generating unit includes a coil part and a drive control part, the coil part is capable of surrounding the first actuator or the second actuator, and two ends of the coil part are electrically connected to the drive control part.

In one preferred embodiment, when the coil part is capable of surrounding the first actuator, the first actuator is a metal rod, while the second actuator is a permanent magnet.

In one preferred embodiment, when the coil part is capable of surrounding the second actuator, the first actuator is a permanent magnet while the second actuator is a metal rod.

In one preferred embodiment, the sleeve further includes at least one operating member, and at least one operating member is electrically connected to the power supply unit and the drive control part.

In one preferred embodiment, the drive control part is a driven circuit board.

In one preferred embodiment, the sleeve further includes an observation window, the position of the observation window corresponds to an installation position of the vial within the channel of the sleeve.

In one preferred embodiment, the magnetic force-driven injection apparatus further includes a rubber stopper, the rubber stopper is disposed between the vial and the second actuator.

In one preferred embodiment, the sleeve further includes a guide member, and the second actuator further includes at least one corresponding guide member, when the second actuator moves relative to the sleeve, the second actuator is moved along the axis by cooperation of at least one corresponding guide member with at least one guide member.

The magnetic force-driven injection apparatus of the present invention generates magnetic force by supply current to the magnetic force-generating unit, so that the magnetic attraction or the magnetic repulsion is generated between the first actuator and the second actuator, so the second actuator is driven either toward the vial closely to activate the injection or farther away from the vial after the injection. Once the direction of the current supplied to the magnetic force-generating unit changes, the moving direction of the second actuator also changes, so the medicine injection or replacement of the magnetic force-driven injection apparatus of this present invention can be easily performed, and it can also effectively simplify the structural design of various components within the magnetic force-driven injection apparatus, improving the ease of assembly.

### BRIEF DESCRIPTION OF THE DRAWINGS

FIG. 1 is a schematic diagram of appearance of the magnetic force-driven injection apparatus of the present invention.

FIG. 2 is a cross-sectional view of the first embodiment of the magnetic force-driven injection apparatus of the present invention.

FIG. 3 is a schematic diagram of performing injection by using the first embodiment of the magnetic force-driven injection apparatus of the present invention.

FIG. 4 is a schematic diagram of completing injection by using the first embodiment of the magnetic force-driven injection apparatus of the present invention.

FIG. 5 is a cross-sectional view of the second embodiment of the magnetic force-driven injection apparatus of the present invention.

FIG. 6 is a schematic diagram of performing injection by using the second embodiment of the magnetic force-driven injection apparatus of the present invention.

FIG. 7 is a schematic diagram of completing injection by using the second embodiment of the magnetic force-driven injection apparatus of the present invention.

### DETAILED DESCRIPTION OF THE PREFERRED EMBODIMENT

Since the various aspects and embodiments are merely illustrative and not limiting, those skilled in the art may, after reading this specification, conceive other aspects and embodiments without departing from the scope of the present invention. The following detailed description and claims will further highlight the features and advantages of these embodiments.

In the disclosure of the present invention, the terms “a” or “an” are used to describe the components and elements disclosed herein. This is done solely for the sake of convenience and to provide a general meaning to the scope of the present invention. Therefore, unless explicitly stated otherwise, such descriptions should be understood to include one or at least one, and the singular form also encompasses the plural.

In the disclosure of the present invention, terms such as “first” or “second,” and similar ordinal numbers are primarily used to distinguish or reference identical or similar components or structures and do not necessarily imply any spatial or temporal order of these components or structures. It should be understood that, in certain cases or configurations, the ordinal terms can be used interchangeably without affecting the implementation of the present invention.

In the disclosure of the present invention, terms such as “comprising,” “having,” or any other similar expressions are intended to encompass non-exclusive inclusivity. For example, a component or structure containing multiple elements is not limited to the elements explicitly listed herein but may also include other elements that are not explicitly mentioned but are inherently typical of such a component or structure.

In the disclosure of the present invention, the term “vial” refers to a component designed to contain medication for performing injection procedures. The vial may be a simple container for storing medication or a container that can be combined with an injection needle or other components. By using a plunger or similar mechanism to press the medication inside the vial, the medication can be delivered through the injection needle into the human body to complete the injection process.

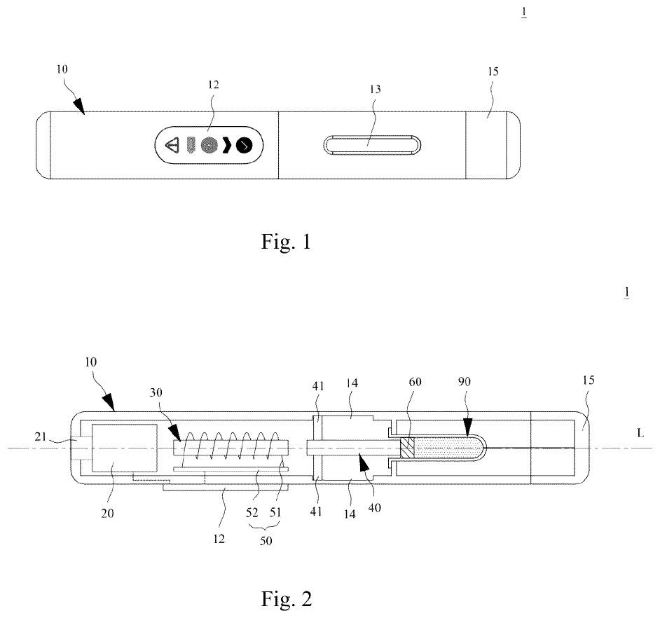

The magnetic force-driven injection apparatus of the present invention is designed to perform an injection operation by being installed with a vial. Please refer to FIG. 1 and FIG. 2, in which FIG. 1 is a schematic diagram of appearance of magnetic force-driven injection apparatus of the present invention, and FIG. 2 is a cross-sectional view of the first embodiment of the magnetic force-driven injection apparatus. As shown in FIG. 1 and FIG. 2, the magnetic force-driven injection apparatus 1 of the present invention includes a sleeve 10, a power supply unit 20, a first actuator 30, a second actuator 40 and a magnetic force-generating unit 50. The sleeve 10 includes a channel 11, the channel 11 is relative to an axis L. The channel 11 is provided for installing the necessary parts of the apparatus, and a movable part is disposed within the channel that can move along the axis L corresponds to the sleeve 10. The vial 90 is arranged on one end of the channel 11 within the sleeve 10. The sleeve 10 is made of durable materials, such as plastic, metal, or alloy, however, the invention is not limited thereto.

In one embodiment of the present invention, the sleeve 10 further includes an observation window 13. The observation window 13 is disposed on the outer surface of the sleeve 10, the position of the observation window 13 corresponds to an installation position of a vial 90 within the channel 10 of the sleeve 10. Therefore, the user can confirm the remaining volume of the medicine inside the current vial 90 through the observation window 13 to decide whether to replace the vial 90 or replenish the medicine. In addition, in one embodiment of the present invention, the sleeve 10 further includes a cover 15. The position of cover 15 is disposed corresponds to the position of the needle on the vial 90. Accordingly, when the magnetic force- driven injection apparatus 1 of the present invention is not in use, the cover 15 can shield the needle on the vial 90, preventing the user from accidentally touching it.

The power supply unit 20 is combined with the sleeve 10to supply the m[power required by each components in the magnetic force-driven injection apparatus 1 of the present invention. Based on different structural design, the power supply unit 20 can arrange directly within the channel 11 of the sleeve 10, for example, the power supply unit 20 is arranged at another end of the channel 11 away from the vial 90, or the power supply unit 20 can arrange outside the channel 11 independently. In one embodiment of the present invention, the power supply unit 20 may be a rechargeable battery module, and power supply unit 20 can include a charging port 21. The charging port 21 is exposed the sleeve 10, so the charging port 21 can connect to a household socket or mobile power source through the transmission line to perform a charging operation on the power supply unit 20.

In addition, in one embodiment of the present invention, the sleeve 10 further includes an operating member 12, and the operating member 12 is electrically connected to the power supply unit 20. The user can switch the power supply status of the power supply unit 20 through the operating member 12 to activate or turn off the magnetic force-driven injection apparatus 1, or to switch, execute, or instruct corresponding functions. The operating member 12 may be consist of a simple button, be a touch panel for user operation, or be a combination of both.

Both the first actuator 30 and the second actuator 40 are disposed within the channel 11 of the sleeve 10, the second actuator 40 is disposed between the first actuator 30 and the vial 90. Both the first actuator 30 and the second actuator 40 are rod-shaped member(s). In the design of the actuation, the second actuator 40 can move toward vial 90 closely or away from it, depending on the direction of movement.

The magnetic force-generating unit 50 is electrically connected to the power supply unit 20, and the magnetic force-generating unit 50 can combine with the first actuator 30 or second actuator selectively. In one embodiment of the present invention, the magnetic force-generating unit 50 includes a coil part 51 and a drive control part 52, and the two ends of the coil part 51 are electrically connected to the drive control part 52 respectively. The magnetic force-generating unit 50 is electrically connected to the power supply unit 20 through the drive control part 52. The coil part 51 can be a helical coil, so the coil part 51 can surround the first actuator 30 or the second actuator 40 based on different designs. The drive control part 52 can be a driven circuit board for controlling the current direction and magnitude of the current supplied to the coil part 51.

In a first embodiment of the present invention, the coil part 51 is surrounded the first actuator 30, so the magnetic force-generating unit 50 is combined with the first actuator to form an electromagnet, and the second actuator 40 is a push rod. The first actuator 30 is a metal rod (such as an iron rod or a nickel rod), while the second actuator 40 is a permanent magnet.

In one embodiment of the present invention, the operating member 12 is further electrically connected to the drive control part 52 of the magnetic force-generating unit 50. The user can control the drive control part 52 through the operating member 12, for example, changing the current direction and the magnitude of the current supplied to the coil part 51.

In addition, the magnetic force-driven injection apparatus further includes a rubber stopper 60. The rubber stopper 60 is disposed within the channel 11 of the sleeve 10 and disposed between the second actuator 40 and the vial 90. When the second actuator 40 is moved toward the vial 90, it can push against the rubber stopper 60, indirectly driving the medicine inside the vial 90 into the human body to complete the injection. In one embodiment of the present invention, the rubber stopper 60 can be fixed at one end of the second actuator 40, near vial 90, however, the invention is not limited thereto.

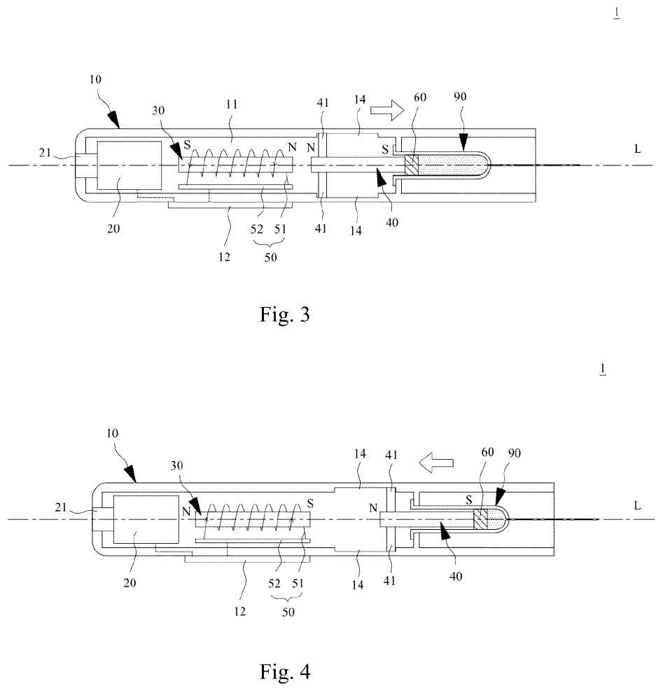

The operating principle of the first embodiment of the magnetic force-driven injection apparatus 1 of the present invention will be explained in detail below. Please refer to FIG. 3 and FIG. 4, in which FIG. 3 is a schematic diagram of performing injection by using the first embodiment of the magnetic force-driven injection apparatus of the present invention, and FIG. 4 is a schematic diagram of completing injection by using the first embodiment of the magnetic force-driven injection apparatus of the present invention.

As shown in FIG. 3 and FIG. 4, when the user wants to use the magnetic force-driven injection apparatus 1 of the present invention to perform the injection, the user can control the power supply unit 20 to start power supply through the operating member 12. The power supply unit 20 provides current to the magnetic force-generating unit 50. When the current is supplied to the coil part 51 through the drive control part 52, an N pole and an S pole are generated at two ends of the first actuator 30, depending on the direction of the current, based on the Ampere's right-hand rule. Meanwhile, with the second actuator 40 designed as a permanent magnet, magnetic attraction or magnetic repulsion is generated between the first actuator 30 and the second actuator 40, thereby, driving the second actuator 40 to move either toward the vial 90 or away from the vial 90.

For example, when the current direction of the coil part 51as shown in FIG. 3, based on the Ampere's right-hand rule, an N pole is generated at the end of the first actuator 30 that is near the second actuator 40, while an S pole is generated at the end of the first actuator 30 that is farther away from the second actuator 40. Meanwhile, with end of the second actuator 40 near the first actuator 30 generates an N pole, and the end farther away generates an S pole. As a result, magnetic repulsion is generated between the first actuator 30 and the second actuator 40, driving the second actuator 40 toward the vial 90 to push the medicine inside the vial 90 and activate the injection.

When the user completes the injection using the magnetic force-driven injection apparatus of the present invention, the current direction can be changed by controlling the drive control unit 52 through the operating member 12. For example, when the current passed through the direction of the coil part 51 as shown in FIG. 4, an S pole is generated at the end of the first actuator 30 that is near the second actuator 40, while an N pole is generated at the end of the first actuator 30 that is farther away from the second actuator 40. Meanwhile, with end of second actuator 40 near the first actuator 30 generates an N pole, while the end farther away generates an S pole. As a result, magnetic attraction is generated between the first actuator 30 and the second actuator 40, driving the second actuator 40 away from the vial 90. Then, the user can remove the used vial 90 and replace new vial 90, so the magnetic force-driven injection apparatus 1 of the present invention can be reused to activate another injection.

Accordingly, the magnetic force-driven injection apparatus 1 of the present invention can easily perform the injection by controlling the magnetic pole changes generated by the current and can also enable repeated injections.

In addition, in the first embodiment of the present invention, the sleeve 10 further includes at least one guide member 14, and the second actuator 40 further includes at least a pair of corresponding guide member 41. When the second actuator 40 moved relatives to the sleeve 10, at least one pair of corresponding guide member 41 cooperates with at least one guide member 14, allowing the second actuator 40 to move along an axis L. Each guide member 14 is a protruding block, and each corresponding guide member 41 is a liner groove designed for the insertion and movement of the protruding block. The liner groove extends in the direction parallel to axis L. The structures of the aforementioned guide member 14 and each corresponding guide member 41 are interchangeable. Additionally, the number and position of the guide member 14 corresponds to number and position of each corresponding guide member 41.

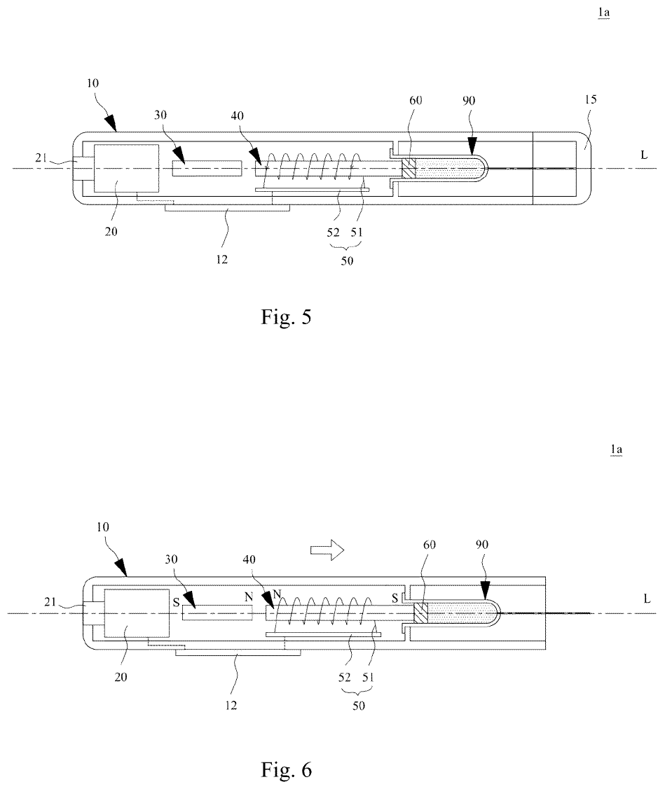

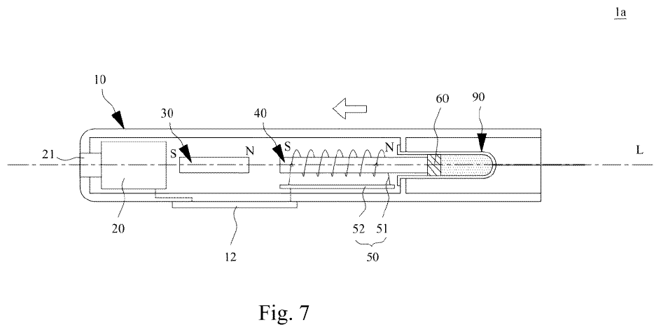

Please refer to FIG. 5 to FIG. 7 together, in which FIG. 5 is a cross-sectional view of the second embodiment of the magnetic force-driven injection apparatus of the present invention, FIG. 6 is a schematic diagram of performing injection by using the second embodiment of the magnetic force-driven injection apparatus of the present invention, and FIG. 7 is a schematic diagram of completing injection by using the second embodiment of the magnetic force-driven injection apparatus of the present invention. The second embodiment of the magnetic force-driven injection apparatus is a variation of the aforementioned first embodiment.

As shown in FIG. 5, in the second embodiment of the magnetic force-driven injection apparatus la of the present invention, the coil part 51 surrounds the second actuator 40, so that the magnetic force-generating unit 50 combines with the second actuator 40 to form an electromagnet, which also serves as the push rod, in which the first actuator 30 is a permanent magnet, and the second actuator 40 is a metal rod (such as an iron rod or a nickel rod).

In can be understood that when the power supply unit 20 supplies current to the coil part 51 through the drive control part 52, with the different current direction, an N pole and an S pole are generated at two ends of the second actuator 40, based on Ampere's right-hand rule. Meanwhile, with the first actuator designed as a permanent magnet, magnetic attraction or magnetic repulsion is generated between the first actuator 30 and the second actuator 40, causing the second actuator 40 and the magnetic force-generating unit 50 to move either toward or away from the vial 90.

For example, when the current passed through the direction of the coil part 51 as shown in FIG. 6, an N pole is generated at the end of the second actuator 40 that is near the first actuator 30, while an S pole is generated at the end of the second actuator 40 that is father away from the first actuator 30. Meanwhile, with end of the first actuator 30 near the second actuator 40 generates an N pole, while the end farther away generates an S pole. As a result, magnetic repulsion is generated between the first actuator 30 and the second actuator 40, causing the second actuator 40 and magnetic force-generating unit 50 to move toward the vial 90, thereby pushing the medicine inside the vial 90 to activate the injection.

After the user completes the injection using the magnetic force-driven injection apparatus of the present invention, the user can change the current direction by controlling the drive control part 52. For example, when the current passed through the direction of the coil part as shown in FIG. 7, an S pole is generated at the end of the second actuator 40 that is near the first actuator 30, while an N pole is generated at the end of the second actuator 40 that is farther away from the first actuator 30. Meanwhile, the end of the first actuator 30 near the second actuator 40 generates an N pole, while the end farther away generates an S pole. As a result, magnetic attraction is generated between the first actuator 30 and the second actuator 40, causing the second actuator 40 and the magnetic force-generating unit 50 to move toward the first actuator 30, thereby moving farther away from the vial 90.

While the invention has been described in terms of what is presently considered to be the most practical and preferred embodiments, it is to be understood that the invention need not be limited to the disclosed embodiments. On the contrary, it is intended to cover various modifications and similar arrangements included within the spirit and scope of the appended claims which are to be accorded with the broadest interpretation so as to encompass all such modifications and similar structures.

1. A magnetic force-driven injection apparatus designed to perform injection operations by being installed with a vial, the magnetic force-driven injection apparatus comprising:
a sleeve, the sleeve includes a channel;
a power supply unit, the power supply unit coupled with the sleeve;
a first actuator, the first actuator is disposed in the channel;
a second actuator, the second actuator is movably disposed in the channel, the second actuator is capable of moving along an axis corresponds to the first actuator; and
a magnetic force-generating unit, the magnetic force-generating unit is electrically connected with the power supply unit, the magnetic force-generating unit is selectively coupled with either the first actuator or the second actuator;
wherein the power supply unit providing the current to the magnetic force-generating unit, a magnetic attraction or a magnetic repulsion is generated between the first actuator and the second actuator, so the second actuator is driven either toward the vial closely to execute the injection or farther away from the vial after the injection.

2. The magnetic force-driven injection apparatus according to claim 1, wherein the magnetic force-generating unit includes a coil part and a drive control part, the coil part is capable of surrounding the first actuator or the second actuator, and the two ends of the coil part are electrically connected to the drive control part.

3. The magnetic force-driven injection apparatus according to claim 2, wherein when the coil part is capable of surrounding the first actuator, the first actuator is a metal rod, while the second actuator is a permanent magnet.

4. The magnetic force-driven injection apparatus according to claim 2, wherein when the coil part is capable of surrounding the second actuator, the first actuator is a permanent magnet, while the second actuator is a metal rod.

5. The magnetic force-driven injection apparatus according to claim 2, wherein the sleeve further includes at least an operating member, and the operating member is electrically connected to the power supply unit and the drive control part.

6. The magnetic force-driven injection apparatus according to claim 2, wherein the drive control part is a driven circuit board.

7. The magnetic force-driven injection apparatus according to claim 1, wherein the power supply unit includes a charging port, and the charging port is exposed outside the sleeve.

8. The magnetic force-driven injection apparatus according to claim 1, wherein the sleeve further includes an observation window, the position of the observation window corresponds to an installation position of the vial within the channel of the sleeve.

9. The magnetic force-driven injection apparatus according to claim 1, further comprising a rubber stopper, the rubber stopper is disposed between the vial and the second actuator.

10. The magnetic force-driven injection apparatus according to claim 1, wherein the sleeve further includes a guide member, and the second actuator further includes at least one corresponding guide member, when the second actuator moves relative to the sleeve, the second actuator is moved along the axis by cooperation of at least one corresponding guide member with at least one guide member.
